(19) United States
(12) Patent Application Publication
(10) Pub. No.: US 2023/0365313 A1
(43) Pub. Date: Nov. 16, 2023

(54) Bag System Containing a Nutrient Composition Comprising Whole Food Sole Source Meal

(71) Applicant: Kate Farms, Inc., Santa Barbara, CA (US)

(72) Inventors: Maia C. Vernacchia, Santa Barbara, CA (US); Vanessa Laura Millovich, Hemet, CA (US); Joseph T. Rupnow, Santa Barbara, CA (US); Dwan Charissa Newman, Sugar Land, TX (US); Erin C. Lamb, Santa Barbara, CA (US); Zhaozhi Huang, Bellevue, WA (US)

(21) Appl. No.: 18/195,761

(22) Filed: May 10, 2023

Related U.S. Application Data
(60) Provisional application No. 63/340,611, filed on May 11, 2022.

(51) Int. Cl. B65D 75/00; A23L 33/12; A23L 33/22; A23L 33/155; A23L 33/15; A23L 33/16; A23L 33/185; A23L 33/105; B65D 41/04; B65D 75/58; A61K 31/202; A61K 36/899; A61K 36/70; A61K 38/16; A61K 33/42; A61K 31/14; A61K 33/10; A61K 31/375; A61K 33/14; A61K 31/015; A61K 31/197; A61K 31/593; A61K 31/355; A61K 31/07; A61K 31/4188; A61K 31/555; A61K 33/34; A61K 31/714; A61K 31/4738; A61K 33/32; A61K 31/455; A61K 31/122; A61K 33/18; A61K 31/4415; A61K 31/525; A61K 33/00; A61K 33/04; A61K 31/51; A61K 31/7004; A61K 31/198; A61K 31/405; A61K 36/73; A61K 36/21; A61K 36/31; A61K 36/45; A61K 36/42; A61K 36/23; A61K 36/48; A61K 36/8945; A61K 36/88; A61K 36/22; A61K 36/54; A61K 36/906; A61K 36/53; A61K 31/23; A61K 31/145

(52) U.S. Cl. CPC B65D 75/008; A23L 33/12; A23L 33/22; A23L 33/155; A23L 33/15; A23L 33/16; A23L 33/185; A23L 33/105; B65D 41/04; B65D 75/5883; A61K 31/202; A61K 36/899; A61K 36/70; A61K 38/168; A61K 33/42; A61K 31/14; A61K 33/10; A61K 31/375; A61K 33/14; A61K 31/015; A61K 31/197; A61K 31/593; A61K 31/355; A61K 31/07; A61K 31/4188; A61K 31/555; A61K 33/34; A61K 31/714; A61K 31/4738; A61K 33/32; A61K 31/455; A61K 31/122; A61K 33/18; A61K 31/4415; A61K 31/525; A61K 33/00; A61K 33/04; A61K 31/51; A61K 31/7004; A61K 31/198; A61K 31/405; A61K 36/73; A61K 36/21; A61K 36/31; A61K 36/45; A61K 36/42; A61K 36/23; A61K 36/48; A61K 36/8945; A61K 36/88; A61K 36/22; A61K 36/54; A61K 36/906; A61K 36/53; A61K 31/23; A61K 31/145; B65D 2207/00; B65D 2575/586

(57) ABSTRACT

A bag system for containing a nutritional composition includes a pouch for holding a nutritional composition front and rear panels which are welded to each other by a bottom weld and a top weld and two welds on each opposed side; and a port for filling and. dispensing for feeding at the top of the pouch with a cap/lid to be resealable. An 8 French tube or larger is connected at the port for delivering the composition without dilution and via enteral pump delivery, A nutritional composition has fatty acids; dietary fibers; carbohydrates; a vitamin and mineral blend; proteins; and a whole food blend. A nutritionally complete nutrient composition for enteral feeding comprises a whole food blend and additional nutrients. Preferably; the nutrient composition is provided in a squeezable serving pouch. The nutrient composition is free of major allergens.

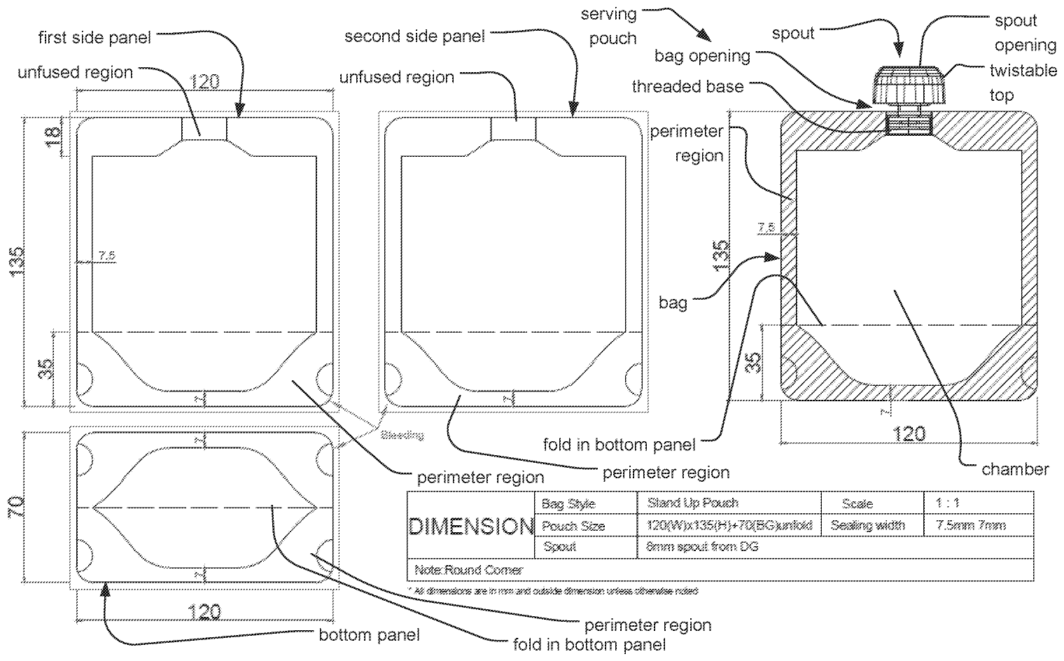

### RELATED APPLICATIONS

This application claims the benefit under 35 USC § 119(e) of U.S. Provisional Application No. 63/340,611, filed on May 11, 2022, which is incorporated herein by reference in its entirety.

### BACKGROUND OF THE INVENTION

Liquid nutrition supplements are well-known and have been readily available in stores and otherwise distributed for medical purposes for many years. Over the past five years there has been an introduction of commercially available blended formulas reflecting whole or pureed foods in a format that can be consumed by individuals with limitations in routine use or with feeding issues.

These compositions come in various flavors and can be consumed in addition to a regular diet; as a replacement for meals in a day, or in lieu of other food altogether. Initially, they were developed mostly for use under medical supervision by those requiring nutrition support i.e.: elderly, patients recovering from illness, and others who need help gaining or retaining weight or otherwise meeting their nutritional requirements, primarily ted by feeding tube. In addition, blenderized formulas have also been targeted and designed for children ages one year and up.

In the medical field, these compositions are used meet the nutritional needs of adult and pediatric patients who have difficulty ingesting, digesting, and absorbing a regular oral diet. These individuals may also have difficulty meeting nutritional goals due to the underlying condition or a co-morbidity, for example: gastroesophageal reflux, or rumination; malabsorption due to disorders such as short bowel syndrome, jejunostomy, EOE (eosinophilic esophagitis), celiac disease, cystic fibrosis, Crohn's Disease or disaccharidase deficiency; endocrine dysfunction resulting from conditions such as diabetes mellitus and proteinuria; increased energy requirements of diseases such as bronchopulmonary dysplasia, cystic fibrosis, or hyperthyroidism; and increased energy needs due to trauma, post-operative recovery, nutritional treatment before, during and after cancer treatment, and during times of metabolic stress as seen with neurological disorders such as cerebral palsy and amyotrophic lateral sclerosis (ALS).

In children, the above may result in malnutrition or Failure to Thrive (“FIT”). Approximately 3-5% of all children admitted to tertiary care centers, and 1% of all children admitted to any hospital, are diagnosed with above ailments or the like. Pediatric manufactured nutritional liquids and powders comprising a targeted selection of nutritional ingredients are well known and widely available, some of which may provide a sole source of nutrition, while others may provide a supplemental source.

Traditional commercially available formulas are often thin in viscosity, contain one or more of the top 9 food allergens, and consist of non-nutritive sweetener ingredients that consumers, HCPs, and patients prefer to avoid. Use of Blended Formulas (BF) in the pediatric population has been on the rise with market research data finding most caregivers being interested in this option. There are several purported clinical benefits of using blenderized tube feeds (BTF) including decreased reflux and gagging, through a perceived mechanism related to optimal viscosity for the patient. In addition, caregivers report a desire to offer real food from ingredients found in BTF commercially available products. With the increased demand from consumers, there are now several commercially prepared formulations available,

However, a major barrier to use of BFs remains physician perspective on ease of use and safety. Much of this perception likely arose from the preparation of homemade blends which may come with increased variation in viscosity and large suspended particles that may lead to tube occlusions. There are now several studies evaluating the IDDSI level and viscosity of several commercial blends and sample homemade blends. These studies provide insight into the relative thickness but do not address the frequency of occlusion events or time spent in preparation and handling of simulated feeding events. Within the pediatric population, market research has identified that 80% of BF users take the formula by tube with 80% of those using pump assisted feedings, 41% administering as a bolus via syringe and 7% by gravity feeding method. Most cited barriers to use by clinicians included: Tube occlusions (59.7%) and increased preparations and handling (39.8%). Eustace, K., Cole, L., & Rollaway, L. (2021). Attitudes and Perceptions of Blenderized Tube Feed Use Among Physicians and Advanced Practice Providers. PEN. Journal of parenteral and enteral nutrition, 10.1002/jpen.2069. Advance online publication. https://doi.org/10.1002/jpen.2069

Current formulations may be considered thicker in consistency and may be difficult to administer in both the in-patient and home setting. The number one barrier to use as cited by clinicians is ease of use with unclear dilution requirements to have the formula flow through tube feeding equipment. Current feeding pumps are often unable to correctly administer and dose the prescribed volume with recent data showing that as much as 40% is undelivered.

### SUMMARY OF THE INVENTION

A bag system for containing a commercially available BF nutritional composition includes a sterile pre-filled pouch filled with a nutritional composition for direct connection of enteral feeding devices via port. Front and rear panels which are welded to each other by a bottom weld, a top weld, and two welds on each opposed side; and a port for filling and dispensing for feeding between the two welds near to the bottom weld and above the bottom weld. In another embodiment, the port is at the top of the pouch, The port is meant to connect directly to an extension set, spike-set, bo-link adapter, or used to empty the contents of the pouch into another vessel for consumption. Several varying feeding tubes with varying diameters (FR sizes) can be used to deliver the composition, without dilution or alteration of the composition. The smallest diameter of enteral feeding device where the composition and the bag can successfully deliver the composition without dilution is an 8 FR size NG tube via enteral feeding pump. The port can also be directly connected to enteral feeding devices to allow for feedings via gravity method. A nutritional composition has fatty acids; dietary fibers; carbohydrates; a vitamin and mineral blend; proteins; and a whole food blend, without the use of the top nine food allergens.

Food processing techniques are often seen as less nutritious than its “Whole Food” counterpart. “Blenderized,” or pureed meals are minimally processed such that the food that is ultimately consumed is relatively similar in composition to the original source of the food (e.g., a vegetable that is eaten raw or prepared in a manner that does not substantially change the nutritional components of the original raw vegetable).

Medical nutrition therapy for many adult and pediatric patients includes blended food formulas which provide the benefit from consuming more whole blended or pureed foods and less processed foods (if any). Some patients also have dietary restrictions or preferences that favor the exclusion of specific ingredients or allergens and the inclusion of whole blended or pureed foods as part of their nutritional intake.

Absorption, distribution, metabolism and excretion. (ADME) studies show the benefits of a more viscous product of at least 200 cP. The viscosity helps for oral/squeeze usage.

At the same time, patients being treated with liquid nutrition and more specifically blenderized food compositions as their sole source of nutrition or any individuals who have difficulty consuming solid foods would benefit from having more options with different flavors, textures, and combinations of nutrients that more closely simulate the experience and nutritional benefits of consuming different types of meals. Moreover, the blenderized food formulas contain naturally occurring plant-based compounds as well as flavors that are close to and are valued as key components found in a regular diet. The blenderized formula offers a potential benefit for the user or consumer to have a feeding experience closer to having whole-food based meals, without ingesting the top nine food allergens.

Accordingly, the presently disclosed nutrient composition comprises a whole food blend, providing a source of nutrition in liquid form with minimally processed whole food ingredients. The whole food blend comprises pureed fruits, vegetables, and/or grains, including apples, beets, broccoli, blueberries, butternut squash, carrots, garbanzo beans, spinach, strawberries, purple sweet potatoes, zucchini, kale, oats or oat flour, bananas, brown rice or rice flour, quinoa or quinoa powder, buckwheat or buckwheat flour, mangos, raspberries, cinnamon, cumin, ginger extract, and/or oregano extract, to name a few examples.

Preferably, the nutrient composition is a nutritionally complete enteral blenderized medical food formula that can be used as a sole source or supplemental source of nutrition for adult and child patients and/or healthy individuals. Thus, the nutrient composition further comprises additional nutrients like protein (e.g., Pisum sativum yellow pea protein or hydrolyzed yellow pea protein), fatty acids (e.g., lipids), dietary fibers, carbohydrates, and vitamins and minerals. Preferably, the nutrient composition comprises a balanced fat blend (e.g., sunflower, flaxseed, medium chain triglycerides). The nutrient composition is free of major allergens (milk, wheat, soy, eggs, peanuts, tree nuts, fish, shellfish, sesame), corn-derived carbohydrates, gluten, sugar alcohols, and non-nutritive sweeteners such as aspartame. The ingredients are plant-based, organic, and free from pesticides, herbicides, artificial fertilizers, antibiotics, or hormones.

The nutrient composition is provided in blenderized liquid form for enteral feeding (e.g., tube and/or oral feeding) and preferably has a pleasing taste and can be taken orally. Generally-, the viscosity of the composition is between 200-2,000 centipoise (cP). In one example, the nutrient composition is packaged in a squeezable pouch or bag, which comprises a spout for ejecting the nutrient composition when pressure is applied to the pouch (e.g., by squeezing the pouch or sucking on the spout). The spout can be opened and closed as desired during consumption of the composition.

Moreover, the nutrient composition is preferably provided in a variety of different “meals” or flavors, which are based on different combinations of ingredients that provide or simulate the taste of the desired “meal.” The meals have been formulated to support digestive system tolerance, with a mild flavor, and targeted for the liking or palate of children. In examples, in addition to other base nutritional ingredients, a first nutrient composition meal variety comprises a blend of bananas, blueberries, zucchini, and oat flour, a second nutrient composition meal variety comprises a blend of mangos, strawberries, zucchini, and oat flour, and a third nutrient composition meal variety comprises a blend of butternut squash, carrot, purple sweet potato, apples, beets, and buckwheat flour.

In general, according to one aspect, the invention features a nutrient composition for nutrition support, the nutrient composition comprising fatty acids, dietary fibers, carbohydrates, a vitamin and mineral blend, proteins, and a whole food blend.

In general, according to another aspect, the invention features a nutrient composition for nutrition support, the nutrient composition comprising sole source or supplemental nutritional components and a whole food blend. The nutrient composition is provided in a squeezable serving pouch.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

The FIGURE is an illustration of a serving pouch for a nutrient composition, according to an embodiment of the invention.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Percentages are assumed to be by weight unless specifically identified to be something else.

The present invention relates to nutrition support that is appropriate for patients, such as children ages 1-13 years who have difficulty digesting foods and have decreased nutrient intake or individuals who have difficulty consuming solid foods. Accordingly, a nutrient composition is disclosed. Nevertheless, the invention is also valuable for healthy individuals.

In general, the nutrient composition comprises a whole food blend, providing a source of nutrition in liquid or blenderized form with minimally processed whole food ingredients. The whole food blend comprises pureed fruits, vegetables, and/or grains, including apples, beets, broccoli, blueberries, butternut squash, carrots, garbanzo beans, spinach, strawberries, purple sweet potatoes, zucchini, kale, oats or oat flour, bananas, brown rice or brown rice flour, quinoa or quinoa powder, buckwheat or buckwheat flour, mangos, raspberries, cinnamon, cumin, ginger extract, and/or oregano extract, to name a few examples.

The nutrient composition is provided in liquid or more specifically blenderized form for enteral feeding, including tube and/or oral feeding, and can be used as a sole source of nutrition or supplemental source of nutrition. As such, the nutrient composition further comprises additional nutrients like protein (e.g., pea protein or hydrolyzed pea protein), fatty acids (e.g., lipids), dietary fibers, carbohydrates, and vitamins and minerals. Preferably, the nutrient composition comprises a balanced fat blend (e.g., sunflower, flaxseed, medium chain triglycerides), pureed fruits, vegetables, and/or grains, including apples, beets, broccoli, blueberries, butternut squash, carrots, garbanzo beans, spinach, strawberries, purple sweet potatoes, zucchini, kale, oats or oat flour, bananas, brown rice or rice flour, quinoa or quinoa powder, buckwheat or buckwheat flour, mangos, raspberries, cinnamon, cumin, ginger extract, and/or oregano extract, to name a few examples. The nutrient composition is free of major allergens (milk, wheat, soy, eggs, peanuts, tree nuts, fish, shellfish, sesame), corn-derived carbohydrates, gluten, sugar alcohols, and artificial sweeteners such as aspartame. The ingredients are plant-based, organic, and free from pesticides, herbicides, artificial fertilizers, antibiotics, or hormones, and the nutrient composition is compatible with a vegan diet.

Moreover, the nutrient composition is preferably provided as a variety of different “meals” or flavors, which are based on different combinations of ingredients that provide or simulate the taste, texture, and/or nutritional components of the desired “meal”.

one example, in addition to other base nutritional ingredients, a first nutrient composition meal variety comprises a blend of bananas, blueberries, zucchini, and oat flour. More specifically, the first formula according to one embodiment comprises approximately 57% purified water, but more generally, purified water could be in a range of 54-61%, or possibly in the range of 47-67%, approximately 2,8% pea protein, but more generally, pea protein could be in a range of 2.5-3.1%, or possibly in the range of 1.5-4.1%, approximately 2.0% high linoleic sunflower oil, but more generally, high linoleic sunflower oil could be in a range of 1.8-2.2%, or possibly in the range of 1.5-2,5%, approximately 0.85% flaxseed oil, but more generally, flaxseed oil could be in a range of 0.64-1.04% or possibly in the range of 0.34-1.34%, approximately 0.85% MCT oil, but more generally, MCT oil could be in a range of 0.64-1.04%, or possibly in the range of 0.34-1.34%, approximately 0.86% vitamin and mineral blend, but more generally, the vitamin and mineral blend could be in a range of 0.7-1.0%, or possibly in the range of 0.6-1.2%, approximately 3.7% blueberries, but more generally, blueberries could be in a range of 2.2-5.2%, or possibly in the range of 0.7-6.7%, approximately 3% zucchini, but more generally, zucchini could be in a range of 2-4%, or possibly in the range of 1-5%, approximately 8.3% whole oat flour, but more generally, whole oat flour could be in a range of 6.3-10.3%, or possibly in the range of 4.3-12.3%, and approximately 20% banana puree, but more generally, banana puree could be in a range of 18-23%, or possibly in the range of 15-25%.

In another example, in addition to other base nutritional ingredients, a second nutrient composition meal variety comprises a blend of mangoes, strawberries, zucchini, and oat flour. More specifically, the second formula according to one embodiment comprises approximately 45% purified water, but more generally, purified water could be in a range of 41-48%, or possibly in the range of 35-55%, approximately 2.5% pea protein, but more generally, pea protein could be in a range of 2.2-2.8%, or possibly in the range of 1.2-3.8%, approximately 1.85% high linoleic sunflower oil, but more generally, high linoleic sunflower could be in a range of 1.65-2.05%, or possibly in the range of 1.35-2.35%, approximately 0.85% flaxseed oil, but more generally, flaxseed oil could be in a range of 0.65-1.05%, or possibly in the range of 0.35-1.35%, approximately 0.85% MCT oil, but more generally, MCT oil could be in a range of 0.65-1.05%, or possibly in the range of 0.35-1.35%, approximately 0.86% vitamin and mineral blend, but more generally, the vitamin and mineral blend could be in a range of 0.71-1.1%, or possibly in the range of 0.56-1.16%, approximately 15% strawberries, but more generally, strawberries could be in a range of 12,5-17,5%, or possibly in the range of 10-20%, approximately 3% zucchini, but more generally, zucchini could be in a range of 2-4%, or possibly in the range of 1-5%, approximately 9.4% whole oat flour, but more generally, whole oat flour could be in a range of 7.4-11.4%, or possibly in the range of 5.4-13.4%, and approximately 21% mango puree, but more generally, mango could be in a range of 18.5-23.5%, or possibly in the range of 16-26%.

In another example, in addition to other base nutritional ingredients, a third nutrient composition meal variety comprises a blend of beets, apples, butternut squash, carrot, purple sweet potatoes, and buckwheat flour. More specifically, the third formula according to one embodiment comprises approximately 62% purified water, but more generally, purified water could be in a range of 58-66%, or possibly in the range of 50-74%, 2% pea protein, but more generally, pea protein could be in a range of 1.7-2,3%, or possibly in the range of 0.7-3.3%, 2.1% high linoleic sunflower oil, but more generally, high linoleic sunflower oil could be in a range of 1.9-2.3%, or possibly in the range of 1.6-2.6%, 0.9% flaxseed oil, but more generally, flaxseed oil could be in a range of 0.7-1.1%, or possibly in the range of 0.4-1.4%, approximately 0.9% MCT oil, but more generally, MCI oil could be in a range of 0.7-11%, or possibly in the range of 0.4-1.4%, approximately 0.86% vitamin and mineral blend, but more generally, the vitamin and mineral blend could be in a range of 0.7-1%, or possibly in the range of 0.6-1.2%, approximately 1.6% beets, but more generally, beets could be in a range of 0.6-2.6%, or possibly in the range of 0-3.6%, approximately 3.4% purple sweet potato puree, but more generally, purple sweet potato puree could be in a range of 1.9-4.9%, or possibly in the range of 0.4-6.4%, approximately 8.3% butternut squash, but more generally, butternut squash could be in a range of 5.8-10.8%, or possibly in the range of 3.3-13,3%, approximately 3.5% carrots, but more generally, carrots could be in a range of 2-5%, or possibly in the range of 0.5-6.5%, approximately 13% buckwheat flour, but more generally, buckwheat flour could be in a range of 11-15%, or possibly in the range of 9-17%.

For enteral tube feeding, the composition can be administered, directly from the spout or port on the top of the pouch using a spike-set to be administered via enteral pump-assisted feedings down to an 8 French NG-tube diameter. One increment on the French scale is equal to ⅓ millimeter, e.g., 8 Fr tubing is 8×0.33 mm=2.67 mm inside diameter. In one example, the nutrient composition is provided in pouches, each of which contains approximately 250 mL of the composition. In still another example, the composition can be emptied from a pouch into any of the regular feeding bags and infused via pump-assisted feedings down to an 8 French NG-tube diameter, large-bore gravity feeds, via a 60 mL syringe as a gravity or bolus teed using the associated plunger of the syringe. Additionally, the port or spout of the pouch connects directly to the Bolink Small Cap device manufactured by U Deliver Medical. The pouch and the contents within can be used within a 24-hour period, after the seal is broken on the port/spout as the cap allows the pouch to be resealable.

Preferably, however, the nutrient composition is packaged in a squeezable serving pouch, which comprises a spout for ejecting the nutrient composition when pressure is applied to the pouch (e.g., by squeezing the pouch or sucking on the spout). The spout can be opened and closed as desired during consumption of the composition.

The FIGURE is an illustration of the serving pouch and its components, according to one embodiment of the present invention.

In the illustrated example, the serving pouch comprises a bag and a spout.

In general, the bag contains the blenderized nutrient composition. In a closed position, the spout seals the nutrient composition within the bag. In an open position, the spout allows the nutrient composition contained within the bag to be ejected out of the spout (e.g., into the mouth of an individual, for consumption). The bag and spout are configured such that in response to pressure being applied to the bag such as by squeezing the bag in the hand while the spout is in the open position, the nutrient composition ejects from the spout in an amount proportional to the applied pressure, which can be in the form of squeezing the bag or sucking on the spout. On the other hand, the bag and spout are configured such that in response to pressure being applied to the bag while the spout is in the closed position, none of the nutrient composition ejects from the spout.

In some embodiments, the spout is adapted to be connected directly to a spike-set for pump assisted feeding. In general, the composition does not require thinning or dilution with water in order to be administered in a feeding tube.

More particularly, the bag comprises a first side panel, a second side panel, a bottom panel, a perimeter region, a bag opening, and a chamber, and the spout comprises a twistable top, a spout opening, and a threaded base. The panels are formed of a flexible material such as flexible plastic.

The first side panel, second side panel, and bottom panel are secured or fused together to form the bag. Accordingly, the first side panel corresponds to a first side of the bag, the second side panel corresponds to a second side of the bag, and the bottom panel corresponds to a bottom of the bag. Each of the panels respectively comprise a perimeter region that joins and secures to (e.g., fuses with) portions of the perimeter regions of the other panels to form the chamber, which contains the nutrient supplement. The first side panel and second side panel are aligned with respect to each other to form common edges, including a top edge, two side edges, and a bottom edge. The perimeter regions of the side panels along the top edge and the two side edges are secured together or fused to form the perimeter region of the bag along the top edge and two side edges. Additionally, the bottom panel is folded over and interposed between the first side panel and second side panel along a bottom edge and a bottom portion of the two side edges of the aligned first and second panels. The perimeter regions of the three panels are fused together along the bottom portion of the two side edges of the aligned first and second panels, with the first side panel forming a top or first layer, a first half of the folded-over bottom panel forming a second layer, a second half of the folded-over bottom panel forming a third layer, and the second side panel forming a bottom or fourth layer. Along the bottom edge, corresponding perimeter regions of the first side panel and the bottom panel are fused together, and, separately, corresponding perimeter regions of the second side panel and the bottom panel are fused together, but the two resulting layers are not fused to each other. As a result, the folded-over bottom panel of the bag can be unfolded, allowing the bottom of the bag to expand (e.g., to accommodate a volume of liquid within the chamber). Otherwise, in the unfolded position, the bag assumes a flattened form with relatively little volume.

The expandable bottom and flexible panel material of the bag, as described above, provide a mechanism for applying pressure to the bag in order to eject the nutrient supplement through the spout. When the bag is filled with the nutrient supplement, the chamber expands such that the bottom panel assumes its unfolded form and each of the side panels are separated from each other along the bottom edge and in the areas corresponding to the chamber of the bag. Applying pressure to one or more sides of the bag (e.g., by squeezing the bag) effectively reverses this deformation process, causing the sides of the bag to move closer together and the bottom panel to move closer to its folded-over form, reducing the available volume of the bag and ejecting the bag's contents through the spout.

Along the top edge of the bag, a central portion of the perimeter regions of the first and second side panels are left unfused, forming a bag opening, which forms a seal around the hollow, cylindrical base of the spout. The cylindrical top of the spout is inserted into the spout base to form the spout, and a threaded region along an exterior surface of a bottom portion of the spout top interacts with a corresponding threaded region along an interior surface of the spout base to provide an opening and closing mechanism for the serving pouch. Twisting the top in different directions (e.g., clockwise or counter-clockwise) causes the top to move toward or away from the spout base, resulting in a correspondingly greater or lesser portion of the spout top to be inserted into the spout base. In examples, twisting the top in one direction causes the top to move further into the spout base until the top and spout base form a seal that prevents contents of the chamber from ejecting through the spout opening, and twisting the top in the other direction causes the top to move out of the spout base until an opening is formed that allows the contents of the chamber to eject through the spout opening. When the spout is in the open position, applying suction (e.g., from an individual's mouth) at the spout opening causes the contents of the chamber to be ejected through the spout opening.

In the illustrated example, the serving pouch has a width of 120 millimeters (mm) a height of 135 mm, and a depth of 70 mm (in the expanded position with the bottom panel unfolded), a sealing width (e.g., width of the perimeter regions of the panels) ranging from 7 ram to 7.5 mm, and a spout width of 8 mm.

Enteral feeding pumps can be used with the blended pouch. In one embodiment, a suitable pump for assisting in the delivery of a nutrient composition includes Moog EnteraLite Infinity pump, available from Moog Inc., 400 Jamison Road, Elma, New York, 14059 USA. In another embodiment, a simple-to-use, precision enteral feeding pump is Covidien Kangaroo™ Joey Enteral Feeding Pump available from Covidien LLC, 15 Hampshire Street, Mansfield, MA 02048 USA. It is easily programmed to deliver continuous or intermittent feeding. Another suitable pump is an OMNI pump from Cardinal.

The blended pouch can directly connect with a spike set. Examples of suitable spike set embodiments include Covidien/Cardinal 765559 Enteral Feeding Pump Safety Screw Spike Set Kangaroo Joey ENPlus PVC NonSterile, available from Covidien; Enteral Feeding Pump Safety Screw Spike Set with ENFit Connector Infinity Silicone NonSterile ENFit Connector, available from Moog LLC; and Enteral Feeding Pump Safety Screw Spike Set Kangaroo ePump ENPlus PVC NonSterile available from Covidien.

The present pouch directly connects to a spilce set as compared to other pouch products having a tear notch.

A Bolink Small Cap can connect directly to the present pouch and be used with gravity bags. The Bolink Small Cap is convenient for home tube feeders by connecting store bought puree pouches to a feeding tube or extension set. Bolink Small Cap is available from U Deliver Medical LLC, 7900 Excelsior Blvd, Hopkins, MN 55343.

The protein source of the nutrient composition is preferably a plant-based protein source such as pea protein (e.g., intact protein, pea protein isolate, hydrolyzed pea protein, and/or pea protein hydrolysate from organic yellow pea). Pea protein is high in branched chain amino acids lysine and arginine, which, for example, are helpful in promoting wound healing. Thus, the nutritional composition includes naturally occurring arginine for nitric oxide generation and many other essential benefits including toxin elimination, immune system maintenance, and to support nutritional needs of individuals with wounds for faster wound healing, to name a few examples.

Preferably, the nutrient composition comprising the whole food blend provides a complete or near-complete amino acid profile, for example, with a Protein Digestibility Corrected Amino Acid Score (PDCAAS) ranging from 0.95 to 1.0, or more preferably 1.0. The viscosity of the composition is preferably between 200-2,000 centipoise (cP). Although such a viscosity is difficult to achieve with the components in the nutrient composition, the disclosed composition readily flows through a No. 8 French tube.

Preferably, the fatty acids of the nutrient composition are provided via a balanced blend of essential omega-6 (e.g., sourced from high linoleic sunflower oil and/or flaxseed oil) and omega-3 fatty acids (e.g., sourced from flaxseed oil), plus the easily digestible organic medium chain triglycerides (MCTs) (for example, derived from organic coconut oil). The flaxseed oil is a colorless to yellowish oil obtained from the dried, ripened seeds of the flax plant. The high linoleic sunflower oil is a variety of sunflower oil containing nearly 70% polyunsaturated linoleic acid. The flaxseed oil, MCT oil, and sunflower oil contribute to viscosity and improve the mouthful of the composition.

The MCT (Medium-Chain Triglycerides) oil is absorbed faster than long-chain triglycerides and provides a quicker source of energy via the liver. They are often digested well even by those who cannot digest normal, long-chain fatty acids. MCT is comprised of primarily caprylic and capric fatty acids and is used as a source of calories for those with intestinal fat malabsorption such as cystic fibrosis. It has also been used to add calories to other powder and liquid pediatric and adult formulas. Unlike other fats, the absorption of MCT oil benefits the patient because it does not go through the lymphatic system; it is transported directly to the liver where it is metabolized for a source of energy.

In one embodiment, the fatty acids include omega-3 fatty acids, as ALA (alpha-linolenic acid) from flaxseed oil, which is derived from the extremely nutritious and disease-preventing flaxseed. Flaxseed has a high concentration of healthy omega-3s, which are fatty adds associated with healthier brains and hearts, better moods, decreased inflammation, and healthier skin and hair. Flaxseed is the richest plant source of ALA, and flaxseed oil contains 50% to 65% of omega-3 fatty acids in the form of ALA. Moreover, flaxseed oil is low in saturated fatty acids (9%), moderate in monosaturated fatty acids (18%), and rich in polyunsaturated fatty acid (65%). Of all lipids in flaxseed oil, ALA is the major fatty acid ranging from 50% to 65% followed by oleic, linoleic, palmitic and stearic acids, which provides an excellent omega-6: omega-3 fatty acid ratio of approximately 0.3:1. Flaxseed oil is also naturally high in antioxidants like tocopherols. Flaxseed oil provides basic nutrition as well as various health benefits in reducing cancer and cardiovascular diseases, lowering LDL-cholesterol and vasodilatory functions. Flaxseed is emerging as an important functional food ingredient, because of its rich contents of ALA, lignans, and fiber. Flaxseed, fibers, and flax lignans have potential health benefits such as reduction of cardiovascular disease, atherosclerosis, diabetes, cancer, arthritis, osteoporosis, and autoimmune and neurological disorders.

In some examples, the fatty acids include omega-6 fatty acids from high linoleic sunflower oil, which comprises essential omega-6 fatty acid content, including palmitic acid, stearic acid, oleic acid, and linoleic acid. The combination of fatty acids in the body are extremely important to maintain various elements of human health, and sunflower oil could help to maintain the balance. The ratio of fatty acids found in sunflower oil, including a substantial amount of linoleic acid (an omega-6 fatty acid) is optimal for patients. Finding a healthy balance between HDL or good cholesterol and LDL or bad cholesterol is very important to the health of patients. Omega-6 (n-6) polyunsaturated fatty acids (PUFA) in high linoleic sunflower oil (e.g., arachidonic acid (AA)) and omega-3 (n-3) PUFA (e.g., eicosapentaenoic acid (EPA)) are precursors to potent lipid mediator signaling molecules, termed “eicosanoids,” which have important roles in the regulation of inflammation.

The nutrient composition typically is engineered to have an optimal combination and ratio of omega-3 and omega-6 fatty acids, which are essential fatty acids that could not be created by the body and must be obtained from diet. Both are polyunsaturated fatty acids that differ from each other in their chemical structure. There are two critical omega-3 fatty acids, EPA and DHA, that patients require. EPA and DHA are the building blocks for hormones that control immune function, blood clotting, and cell growth as well as components of cell membranes. The body also constructs hormones from omega-6 fatty acids. In general, hormones derived from the two classes of essential fatty acids have opposite effects. Those from omega-6 fatty acids tend to increase inflammation (an important component of the immune response), blood clotting, and cell proliferation, while those from omega-3 fatty acids decrease those functions. Both families of hormones must be in balance to maintain optimum health. Many nutrition experts believe that before processed foods, humans consumed omega-3 and omega-6 fatty acids in roughly equal amounts. Currently, omega-6 foods are numerous in modern diets, including seeds and nuts and the oils are extracted from them. A ratio of omega-6:3 equal to or less than 4:1 may improve overall inflammation and risk for cardiovascular disease.

In general, the vitamin and mineral blend preferably includes any combination of dicalcium phosphate, magnesium chloride, calcium carbonate, choline bitartrate, sodium ascorbate, trisodium citrate, potassium citrate, taurine, L-carnitine, inositol, DL-alpha-tocopheryl acetate, niacinamide, zinc oxide, ferric orthophosphate, calcium pantothenate, pyridoxine hydrochloride, copper sulfate, magnesium sulfate, riboflavin, thiamine hydrochloride, vitamin A palmitate, beta carotene, folic acid, biotin, chromium picolinate, phytonadione, sodium molybdate, sodium selenite, potassium iodide, vitamin D2, cyanocobalamin (vitamin B12), potassium chloride, sodium chloride, ferric pyrophosphate, manganese sulfate, cholecalciferol (Vitamin D3), and phylloquinone. The vitamin and mineral blend also preferably includes amino acids such as L-cysteine, L-threonine, L-tryptophan, L-lysine, and L-leucine.

The vitamin and mineral blend often contains potassium citrate, magnesium citrate, and potassium chloride.

Potassium citrate is an essential nutrient for homeostasis in the body. It works alongside sodium to maintain normal blood pressure. Potassium, which is an electrolyte, helps to maintain a healthy fluid balance in the body. It also helps to transmit electrical pulses to allow proper nerve and muscle function. Potassium citrate is specifically used in some examples to help maintain effective body pH.

The nutrient composition further preferably comprises dipotassium phosphate, which is a highly water-soluble salt that acts is a source of phosphorus and potassium. Dipotassium phosphate commonly comes in the form of a colorless, white substance. Potassium is one of the seven essential macro minerals, of which the human body requires at least 100 milligrams daily in order to sufficiently support its key processes. A healthy intake of potassium can decrease risk of stroke, lower blood pressure, protect against the loss of muscle mass, preserve bone mineral density, and reduce the risk of kidney stones.

The nutrient composition further comprises tricalcium phosphate, in some implementations. This is a calcium salt of phosphoric acid and acts as a source of calcium and phosphorus. Tricalcium phosphate consists of positively charged particles of calcium combined with negatively charged particles called phosphates.

While this invention has been particularly shown and described with references to preferred embodiments thereof, it will be understood by those skilled in the art that various changes in form and details may be made therein without departing from the scope of the invention encompassed by the appended claims.

What is claimed is:

1. A bag system for containing a nutritional composition, comprising:
a pouch for holding a nutritional composition front and rear panels which are welded to each other by a bottom weld and a top weld and two welds on each opposed side; and a port for filling and dispensing for feeding;
a port capable of delivering the composition through an 8 French tube or larger without dilution, wherein said port has a cap allowing the pouch to be resealable; and
a nutritional composition having fatty acids; dietary fibers; carbohydrates; a vitamin and mineral blend; proteins; and a whole food blend.

2. The system according to claim 1, wherein a viscosity of the nutritional composition is between 200-2,000 centipoise (cP).

3. The system according to claim 1, wherein the pouch connects directly to a spike-set for pump assisted feeding.

4. The system according to claim 1, wherein the composition does not require thinning or dilution with water in order to be administered in a feeding tube.

5. The system according to claim 1, wherein the fatty acids of the nutrient composition include a blend of essential Omega-6 and Omega-3 fatty acids; and medium chain triglycerides.

6. The system according to claim 1, wherein the dietary fibers include inherent dietary fibers from fruit and vegetable purees and flours from sources such as oat and buckwheat, and/or pea protein.

7. The system according to claim 3, wherein the carbohydrates include fruit/vegetable purees, oat/buckwheat flours, and pea protein.

8. The system according to claim 1, wherein the vitamin and mineral blend includes a compound selected from one of the following: tricalcium phosphate, dipotassium phosphate, choline bitartrate, calcium carbonate, dimagnesium phosphate, potassium chloride, sodium ascorbate, sodium chloride, beta carotene, calcium pantothenate, cholecalciferol (vitamin D3), DL-alpha-tocopheryl acetate, ferric pyrophosphate, vitamin A palmitate, biotin, chromium picolinate, copper sulfate, cyanocobalamin (vitamin B12), folic acid, manganese sulfate, niacinamide, phylloquinone, potassium iodide, pyridoxine hydrochloride, riboflavin, sodium molybdate, sodium selenite, thiamine hydrochloride, zinc oxide, inositol, 1-cysteine, 1-carnitine, 1-leucine, 1-lysine hydrochloride, 1-threonine, 1-tryptophan, and taurine.

9. The system according to claim 1, wherein the protein is selected from one of the following: Pisum sativum yellow pea protein, hydrolyzed yellow pea protein, intact pea protein, pea protein isolate, hydrolyzed pea protein, and pea protein hydrolysate from organic yellow pea.

10. The system according to claim 1, wherein the whole food blend includes at least one of the following: pureed fruits, vegetables, and grains.

11. The system according to claim 10, wherein the pureed fruits, vegetables, and grains are selected from at least one of the following: apples, beets, broccoli, blueberries, butternut squash, carrots, garbanzo beans, spinach, strawberries, purple sweet potato, zucchini, kale, oats or oat flour, bananas, brown rice or rice flour, quinoa or quinoa powder, buckwheat or buckwheat flour, mangos, raspberries, cinnamon, cumin, ginger extract, and oregano extract.

12. A blenderized nutrient composition for nutrition support, the nutrient composition comprising:
fatty acids; dietary fibers; carbohydrates; a vitamin and mineral blend; proteins; and a whole food blend.

13. The composition according to claim 12, wherein a viscosity of the composition is between 200-2,000 centipoise (cP).

14. A blenderized nutrient composition for nutrition support, the nutrient composition comprising:
sole source or supplemental nutritional components; and
a whole food blend, wherein the nutrient composition is provided in a squeezable serving pouch.

15. The composition according to claim 14, wherein a viscosity of the composition is between 200-2,000 centipoise (cP).

16. The composition according to claim 14, wherein the pouch connects directly to a spike-set for pump assisted feeding.

7. The composition according to claim 14, wherein the composition does not require thinning or dilution with water in order to be administered in a feeding tube.

18. The composition according to claim 14, wherein the composition flows through an 8 French tube without dilution.
